(19) United States
(12) Patent Application Publication
(10) Pub. No.: US 2011/0082373 A1
(43) Pub. Date: Apr. 7, 2011

(54) METHODS AND APPARATUS FOR THE DETECTION OF CARDIOPULMONARY DEFECTS

(76) Inventors: John C. Gurley, Lexington, KY (US); Kevin D. Donohue, Lexington, KY (US)

(21) Appl. No.: 12/793,444

(22) Filed: Jun. 3, 2010

Related U.S. Application Data
(60) Provisional application No. 61/183,971, filed on Jun. 4, 2009.

(51) Int. Cl. A61B 8/06

(52) U.S. Cl. 600/454

(57) ABSTRACT

A diagnostic system for detecting a right-to-left cardiopulmonary shunt in a patient. The system includes at least one Doppler ultrasound transducer, a transducer housing, a transducer housing placement device to affix the Doppler ultrasound transducer housing over a peripheral blood vessel, and a control system configured to receive and process the echoes and transform the echoes into data indicative of the presence or absence of a bubbles within the peripheral blood vessel, thereby determining whether or not the patient has a right-to-left cardiopulmonary shunt. A method of detecting a right-to-left cardiopulmonary shunt in a patient is also provided.

### CROSS REFERENCE TO RELATED APPLICATIONS

This application claims priority to U.S. Provisional Application No. 61/183,971 filed on Jun. 4, 2009, the entire disclosure of which (including the attachments thereto) is incorporated herein by reference.

### BACKGROUND

Detection and characterization of cardiopulmonary defects is difficult and typically requires expensive and complicated testing in a clinical setting under the guidance of skilled clinicians. One cardiopulmonary defect of particular importance that is difficult to diagnostically screen outside of a hospital setting is a right-to-left shunt such as a patent foramen ovale or atrial septal defect. The foramen ovale is a small hole located in the atrial septum that is used during fetal circulation to speed up the travel of blood through the heart. In the womb, a fetus does not use its own lungs to oxygenate blood. Rather, the fetus relies on the mother to provide oxygenated blood from the placenta through the umbilical cord. Since fetal lungs are not needed until birth, fetal blood can travel from the veins to the right side of the fetal heart and cross to the left side of the heart through the foramen ovale and avoid the unnecessary trip to the fetal lungs.

The foramen ovale normally closes soon after birth, but in approximately 1 out of 4 people the opening never closes. If it does not, it is called a patent foramen ovale (PFO). An echocardiogram is typically used to diagnose a PFO. If the PFO is not easily seen, a cardiologist can perform a “bubble test.” Normal saline solution with suspended micro-bubbles of air is injected into a vein as the cardiologist watches the heart on an ultrasound (echocardiogram) monitor. If a PFO exists, the tiny air bubbles will be seen moving from the right to left side of the heart during the test.

There are usually no complications associated with a PFO. Studies do suggest that older patients with PFOs have a higher rate of paradoxical thromboembolic stroke. There is a growing body of evidence that percutaneous closure of patent foramen ovale can achieve a significant reduction in the number of migraine episodes both with and without aura following closure. One study indicates that closure of a large PFO can reduce migraine frequency and severity in patients with subclinical brain lesions, as well as those with prior stroke. The best candidates for PFO closure, based on this study, include patients with a significant permanent shunt, atrial septal aneurysm, coagulation abnormalities, persistent eustachian valve, or refractory migraine with aura.

Another type of right-to-left shunt is an atrial septal defect (ASD). Like a PFO, an ASD permits blood to flow between the right and left atria, through the intraatrial septum. The term ASD is generally applied to defects that involve missing tissue, and therefore ASD's typically exhibit both right-to-left and left-to-right flow between the atria. It should be noted that the term PFO is sometimes used to refer to a small ASD which exhibits bidirectional shunting.

Yet another type of cardiopulmomary defect associated with right-to-left shunts is pulmonary arteriovenous malformation (“PAVM”). PAVM is a rare pulmonary vascular anomaly. Although most patients are asymptomatic, PAVMs can cause dyspnoea. Because of paradoxical emboli, various central nervous system complications have been described including stroke and brain abscess. There is a strong association between PAVM and hereditary haemorrhagic telangiectasia.

Chest radiography and contrast enhanced computed tomography provide initial diagnostic tools for PAVM, but pulmonary angiography is the currently the diagnostic tool of choice. Combined color Doppler ultrasound and amplitude ultrasound angiography are commonly employed non-invasive techniques for diagnosing PAVM and provide an alternative approach to angiography in evaluating the efficacy of embolotherapy.

In an anatomic shunt, such as a PFO, blood moves from one circulatory system to the other through an orifice in the heart or great vessels. Recirculation of blood produces a physiologic shunt, which can exist in the absence of an anatomic shunt, for example, when great vessels are transposed.

The magnitude and direction of shunts vary, both within the cardiac cycle and over time. An example is the normal fall in the left ventricular pressure during diastole preceding the fall of pressure in the right ventricle so that transient right-to-left shunting occurs. Additionally, anesthesia and operative conditions can have an effect on the magnitude and direction of shunting. The most significant determinants of flow through the shunt are the size of the orifice ad the relative outflow resistances on either side of the shunt. Outflow resistance can be determined by systemic vascular resistance, ventricular compliance, pulmonary vascular resistance, and the determination and measurement of anatomic obstructions. Factors influencing shunt flow are given by the Hagen-Poiseuille equation Q=Pπr⁴/8ηL where Q is flow, P is the pressure gradient, r is the radius, η is the fluid viscosity, and L is tube length.

The detection and treatment of right-to-left shunts is important because the recirculation of systemic venous blood can have detrimental effects such as a decrease in arterial pulmonary blood flow with admixture of deoxygenated blood to the systemic circulation producing hypoxemia and cyanosis, and impedance to right ventricular ejection resulting in ventricular pressure overload that ultimately can lead to dysfunction in the right ventricle.

While a variety of devices and techniques may exist for detecting cardiopulmonary defects using Doppler ultrasound techniques, it is believed that no one prior to the inventors has made or used an invention as described herein.

### BRIEF DESCRIPTION OF THE DRAWINGS

While the specification concludes with claims which particularly point out and distinctly claim the invention, it is believed the present invention will be better understood from the following description of certain examples taken in conjunction with the accompanying drawings. In the drawings, like numerals represent like elements throughout the several views.

FIG. 1 is a schematic illustration of a for detecting right-to-left shunts such as PFO's and PAVM's;

FIG. 2 is a schematic view of a portion of the system of FIG. 1, wherein the transducer has been positioned on a patient's wrist;

FIG. 3 is a partial cross-sectional view of the transducer positioned on a patient's wrist;

FIG. 4 is a partial cross-sectional view of another embodiment of a transducer and transducer housing for use with the system of FIG. 1;

FIG. 5 is an end view of another alternative embodiment of a transducer array and housing;

FIG. 6 is an exemplary algorithm for processing Doppler audio output;

FIG. 7 is a sample output from a specularity filter;

FIGS. 8A-8C depict sample Doppler audio output, envelope detection on the audio output, and specularity filter data on the audio output; and

FIGS. 9A-9C depict sample air emboli assessments.

The drawings are not intended to be limiting in any way, and it is contemplated that various embodiments of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The following description of certain examples should not be used to limit the scope of the present invention. Other features, aspects, and advantages of the versions disclosed herein will become apparent to those skilled in the art from the following description, which is by way of illustration, one of the best modes contemplated for carrying out the invention. As will be realized, the versions described herein are capable of other different and obvious aspects, all without departing from the invention. Accordingly, the drawings and descriptions should be regarded as illustrative in nature and not restrictive.

Examples described herein include systems and methods for the detection of PFO's, PAVM's and other cardiopulmonary and circulatory system defects. Systems and methods described herein may even be used outside of the hospital setting, such as in a physician's office, health clinic or diagnostic facility. Doppler ultrasound is utilized to diagnose the existence of cardiopulmonary defects, particularly right-to-left shunts such as PFO's, ASD's and PAVM's.

In one example, a dispersion of microbubbles of air in a solution such as normal saline is used as a contrast agent for Doppler measurements. The dispersion of microbubbles in normal saline (also referred to as agitated saline) may be created, for example, by a commonly-used push-pull technique using two connected syringes. One or more additives such as a surfactant (e.g., the patient's own blood) may be used to modify the surface tension of the bubbles so as to enhance their stability in the solution. When the patient's blood is added to the saline solution it may be added, for example, at a concentration of up to about 10% by volume.

The microbubble dispersion is injected into a blood vessel at any peripheral or central venous location such as a peripheral brachial vein (e.g. the basilica vein, the intermediate antebrachial vein, or the accessory cephalic vein). It should be noted that, while injection into the femoral vein or inferior vena cava (IVC) may be employed, injection into these locations is more difficult outside of a hospital setting. The microbubbles travel through the venous system to the right atrium. In a typical atrial septum, the foramen ovale is closed and the microbubbles will travel to the lungs where the injected microbubbles are filtered out by the lungs. When the foramen ovale has not completely closed (i.e., a PFO is present) or other type of right-to-left inter-atrial shunt exists, a portion of the microbubbles pass directly from the right atrium to the left atrium, bypassing the lungs. In other words, the PFO or other shunt allows for the mixing of oxygenated and non-oxygenated blood through the unclosed hole. Because of this, injected microbubbles will appear in atrial blood. Similarly, while a PAVM is not an intra-atrial right-to-left shunt, a PAVM allows some of the injected microbubbles to pass through the lungs without being absorbed. Thus, a PAVM will also result in injected microbubbles appearing in atrial blood.

While microbubbles in atrial blood can be detected transcranially, their quantity at that situs inhibits the characterization of the shunt or other cardiac defects because of the Doppler ultrasound transducer's inability to resolve individual microbubbles. In addition, transcranial Doppler typically requires expensive equipment and use by highly-experienced end-users (typically in a hospital setting). Systems and methods described herein may be used to detect microbubbles in a peripheral blood vessel such as a brachial artery in order to diagnose the presence of a PFO, ASD, PAVM or other cardiopulmonary defect. As an alternative to a brachial artery, other peripheral arteries may be monitored to detect microbubbles such as a carotid or femoral artery.

During use of the systems and methods described herein, one or more Doppler ultrasound transducers are secured in place against a target blood vessel. The transducer and/or the transducer housing may be adjusted to optimize transmittal of ultrasound to a target blood vessel and/or reception of the Doppler ultrasound echoes from the target blood vessel's blood and blood borne constituents. Doppler data is collected from the transducer(s) and recorded. After baseline data has been acquired, a microbubble dispersion is injected into the patient and additional Doppler data is collected and recorded for a period of time following the injection. Signal data from the time periods before and after injection is processed and filtered, and the resulting data is then analyzed for evidence of microbubbles in the target blood vessel. Algorithms employed by a control system characterize the processed and filtered signal data and provide interpretation of the aforementioned data in the form of potential diagnoses of cardiopulmonary defects such as a PFO, ASD or PAVM.

Since a PFO or other defect can exist in a closed state until stressors are experienced (e.g., a change in thoracic pressure), the testing methods further described herein may be run under stress-free conditions, e.g. normal breathing, as well as under stress-induced conditions, e.g. Valsalva breathing. As further described herein, a pressure transducer may be integrated with a pressure transducer housing (e.g., a mask) used by a patient during the breathing maneuvers, the release of which marks the time for injection of the microbubbles. In other words, the microbubble dispersion may be injected at the beginning of the strain phase of the Valsalva maneuver or other stressor. In some embodiments, the Valsalva maneuver or other stressor is terminated when the microbubbles reach the right atrium. By way of example, the system may provide an audible and/or visual signal at a predetermined time following microbubble injection indicating that the Valsalva maneuver or other stressor should be terminated.

Embodiments of the systems and methods described herein are suitable for use outside of the hospital setting and do not require the immediate involvement of a specialist as no complicated analysis is necessary on the part of the operator to determine the existence or non-existence of a PFO or other defect the control system is programmed to recognize. The test may be minimally invasive, requiring only intravenous injection of an agitated saline solution into a blood vessel (e.g., a brachial vein).

FIG. 1 schematically depicts an exemplary system (10) for detecting right-to-left shunts such as PFO's and PAVM's, as well as other cardiopulmonary system defects. Defect detection system (10) includes at least one ultrasonic transducer (12) which is shown mounted in a housing (14). As further described herein, the system may include a plurality of ultrasonic transducers such as in the form of an array. Housing (14) may be configured to facilitate proper placement and positioning of transducer (12) on a patient. System (10) further includes a control system (16) which transmits signals to transducer (12) causing the transducer (12) to emit ultrasonic waveforms, and also receives ultrasonic echo signals (i.e., Doppler shift signals) from transducer (12). Control system (16) further processes the echo signals received from the transducer (12) in order to determine if a circulatory or cardiopulmonary system defect such as a right-to-left shunt is present in a patient. Defect detection system (10) shown in FIG. 1 further includes one or more input devices (26) (e.g., a keyboard) for supplying user input to control system (16), a display device (28) (e.g., a visual display such as an LCD screen) for displaying information received from control system (16) to a user, one or more speakers (30) for emitting audio signals received from control system (16), external memory (32) (e.g., a storage device) for receiving data from and optionally supplying data to control system (16), and a printer (34) for printing data received from control system (16). It will be understood that one or more of the foregoing devices which receive from and/or transmit data to control system (16) may be omitted. Alternatively, defect detection system (10) may be configured such that a user may connect standard peripheral devices (e.g., keyboard, mouse, printer, display, storage device, etc.) to control system (16) via one or more standard ports such as universal serial bus (USB).

Control system (16) may be configured in any of a variety of ways, and generally includes at least one processor (such as a CPU) and at least one memory for providing instructions to the at least one processor. Control system (16) controls the operation of defect detection system (10) in accordance with preprogrammed instructions as well as user input. In the exemplary embodiment shown in FIG. 1, control system (16) includes a Doppler unit (18) and a signal processing unit (20). Each of these units may include its own processor and memory. Doppler unit (18) is configured to generate ultrasonic waveforms which are then emitted by transducer (12), receive echo signals from transducer (12), and to generate Doppler shift data which is then communicated to signal processing unit (20). In one embodiment, Doppler unit (18) generates Doppler shift data in the form of an audio signal which is then further processed by signal processing unit (20) to determine the presence of a PFO, PAVM, or other circulatory or cardiopulmonary system defect. In one embodiment of a method for detecting a circulatory or cardiopulmonary system defect, Doppler shift data is generated before, during and after the injection of a microbubble dispersion into a patient's peripheral blood vessel such as a peripheral brachial vein, and the Doppler shift data is further processed to determine whether or not a portion of the microbubbles have bypassed the lungs and are therefore present in a blood vessel adjacent transducer (12) (e.g., in a brachial artery).

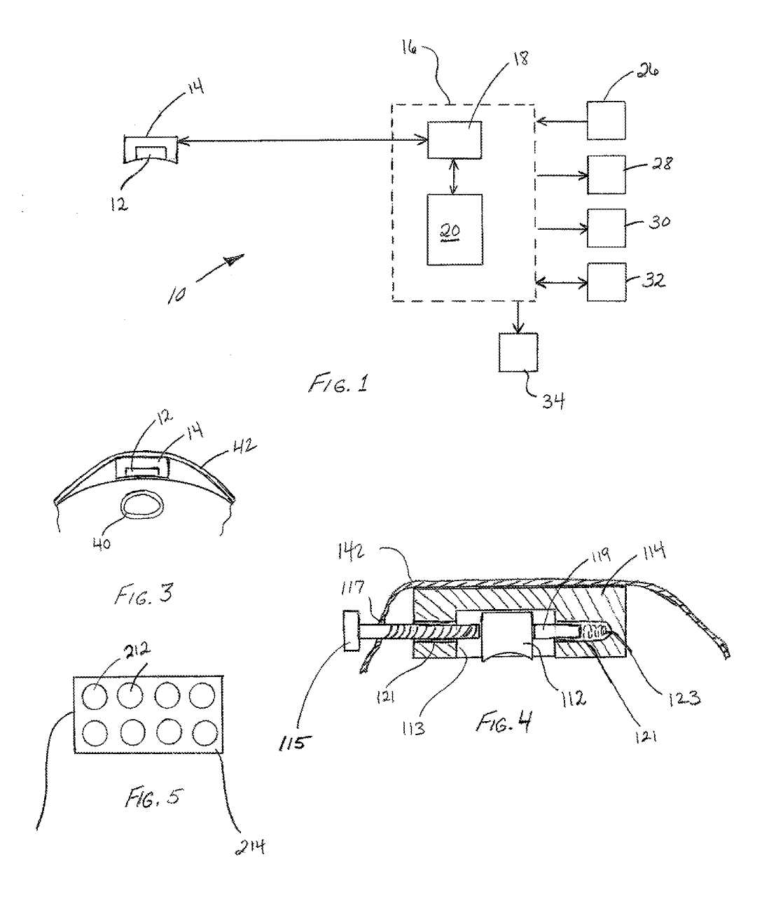

As depicted in FIGS. 2 and 3, the Doppler ultrasound transducer (12) may be positioned over a peripheral artery, such as the radial artery (40), at or near the wrist. This location is suitable for many users since clinicians are familiar with locating a radial pulse. The radial location also permits a stable, hands-free Doppler ultrasound transducer (12) to be secured in place using, for example, a wrist bracelet style transducer fixation device (42). Transducer (12) serves two functions: it provides a Doppler flow signal to facilitate the positioning of the transducer over the target artery; and it transmits amplitude data to the control system which processes the data for the purpose of the identification of microbubbles in the target artery. Alternate locations for the positioning of transducer (12) include other peripheral arteries such as the brachial, ulnar and popliteal arteries. The transducer fixation device (42) may also include an adjustment mechanism, as further described below, which allows the operator to fine-tune the transducer position by, for example, listening to familiar swishing sounds and/or viewing a signal strength display.

In one embodiment, a Doppler ultrasound transducer fixation device (42) comprises a hook and loop fabric strap, as shown in FIG. 2. The transducer housing (14) may be secured to the hook and loop fabric strap by, for example, an adhesive or one or more fasteners. Such a strap permits the clinician to achieve a secure fit of the fixation device about the wrist (or other location) to prevent the Doppler ultrasound transducer (12) from moving from its desired position. The strap allows the bracelet to be tightened or loosened by the clinician and facilitates rapid placement and removal regardless of the size of the patient's wrist.

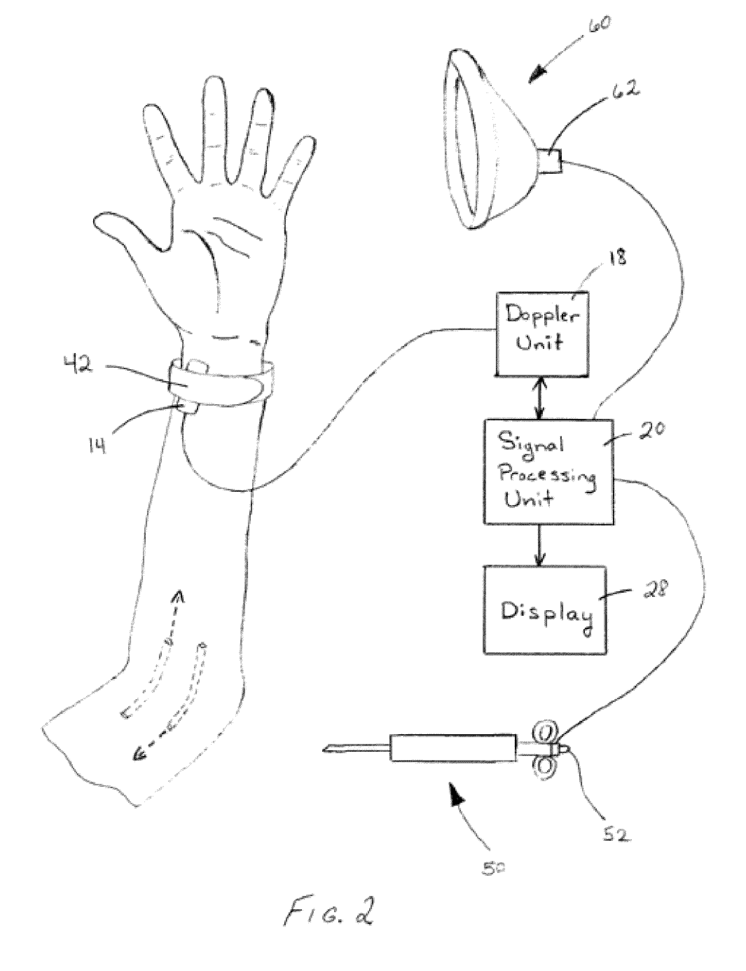

Any of a variety of adjustment mechanisms may be provided for allowing the clinician to adjust the location of the transducer once secured to a patient. For example, as shown in the partial cross-sectional view of FIG. 4, Doppler transducer (112) is slidably mounted within a cavity (113) in transducer housing (114). Cavity (113) is sized so as to retain transducer (112) therein will still allowing transducer (112) to be slidingly adjusted within cavity (113). One or more additional features such as guide rails and the like may be included in order to slidingly retain transducer (112) within cavity (113). Housing (114) is also depicted attached to a fabric strap fixation device (142), as described previously (e.g., by an adhesive or one or more fasteners).

A threaded set screw (115) extends through an aperture (121) in fabric strap (142) and into a threaded bore (121) in housing (114) such that the distal end of the set screw (115) abuts against a portion of transducer (112). A clinician or other end-user may adjust the position of transducer (112) after the transducer (112) has been secured on a patient's wrist by simply turning the set screw (115). This allows the transducer (112) to be adjusted laterally across the patient's wrist in order to ensure optimal positioning with respect to an underlying artery. Proper positioning may even be guided by monitoring a Doppler echo signal, for example, by observing a visual and/or audio indication of signal strength provided by control system (16). A spring loaded pin (119) may also be provided in housing (114), and is depicted abutting against a side of transducer (112) opposite set screw (115). Pin (119) extends into a bore (121) in housing (114), and a spring (123) is positioned within bore (121) in order to spring bias pin (119) towards transducer (112). Spring loaded pin (119) provides resistance so that as the set screw (115) is adjusted, the Doppler ultrasound transducer (112) is tensioned and will slide within cavity (113) of housing (114).

In yet another embodiment, a plurality of Doppler ultrasound transducers are utilized. For example, a dual Doppler ultrasound transducer arrangement allows for the verification of the data from a first Doppler ultrasound transducer by a second Doppler ultrasound transducer and enhances the system's ability to discern true detections from random artifacts.

Yet another alternative embodiment is shown in FIG. 5, wherein a plurality of Doppler ultrasound transducers (212) are mounted in an array in a housing (214). When this embodiment is employed, the housing (214) is simply positioned against the patient's skin in the area adjacent the targeted artery transducers. Control system (16) may be programmed to select the transducer(s) (212) providing the best Doppler echo signal, thus eliminating the need to reposition or adjust an individual Doppler ultrasound transducer. Such an arrangement further facilitates the proper positioning of the Doppler transducer(s) at the radial artery (or other location).

As mentioned previously, the method utilized for the detection of a PFO, ASD, PAVM or other right-to-left shunt includes the step of injecting microbubbles intravenously into a peripheral vein, such as a brachial vein. A dispersion of microbubbles in saline may be formed in any of a variety of ways. By way of example, and as known to those skilled in the art, air may be entrained in a normal saline solution by agitating the solution between two syringes. The saline solution is forced from a first syringe into a second syringe, typically through a ball cock valve. Once a dispersion of microbubbles in saline has been created, one of the syringes may then be used to inject the microbubbles into the a patient's vein.

By way of example only, a 10 cc syringe (50) may be utilized (see FIG. 2). In one embodiment, a typical injection may comprise about 9 cc of normal saline and about 1 cc of air in the form of microbubbles. As mentioned previously, a few drops of the patient's blood or another medically appropriate surfactant may be added to the normal saline in order to modify the surface tension of the fluid and stabilize the microbubbles.

Syringe (50) may also include an actuator such as a switch (52) located, for example, at the top of the plunger. Switch (52) is in communication with control system (16) and, when activated, sends a signal to control system (16) (e.g., to signal processing unit (20)). A clinician may activate the switch (52) when the microbubbles are injected into the patient in order to signal to the control system (16) that injection has commenced (or completed). The control system (16) may use this signal to segment acquired data into pre- and post-injection sets. In addition, as further described herein, the control system (16) may use the injection timing data in conjunction with a timing algorithm in order to provide a visible and/or audible signal that a Valsalva maneuver should be commenced by the patient. After a predetermined period of time, the control system (16) may similarly indicate that the Valsalva maneuver should be stopped (i.e., when the microbubbles are expected to arrive in the right atrium). Communication between switch (52) and control system (16) may be wired or wireless.

As an alternative to a user-activated switch (52) or other actuator, or in addition thereto, an injection sensor may be provided. The injection sensor may be used to sense injection of the microbubble dispersion and send a signal to the control system (16) indicating when injection occurs. Such a sensor can comprise one or more wired or wireless sensors known to those skilled in the art of sensor engineering. A pressure sensitive sensor pad on or within the syringe may be used, for example, or even a sensor capable of optical analysis of the cavity of the syringe are but a few of the many possible varieties of sensors available.

As further discussed herein, Doppler unit (18) causes transducer (12) to emit pulses of ultrasonic waves which are directed into a patient's artery located adjacent transducer (12). Ultrasound reflected from blood cells traveling in the artery (i.e., Doppler echoes) are received by transducer (12) and a signal is sent from transducer (12) to Doppler unit (18). As is known to those skilled in the art, Doppler unit (18) may process the received echo signals and generate, for example, audio output indicative of the Doppler echo (or shift) signals. The audio output (or other form of data output) is then transmitted from Doppler unit (18) to signal processing unit (20) of control system (16). Data may be collected for a period of time prior to microbubble injection in order to compile background data.

Following microbubble injection into a vein, the deoxygenated blood in the vein carries the agitated saline solution with its entrained microbubbles back to the patient's heart via the major veins (i.e. superior and inferior vena cavas) where it enters the right atrium. When a PFO or ASD is present and open, a portion of the blood carrying the injected microbubbles passes directly into the left atrium then into the left ventricle, bypassing the patient's lungs where the microbubbles would normally be removed by the lungs. After the left ventricle, the blood containing the microbubbles is ejected to the aorta for distribution through the arteries. In the case of a PAVM or other non-atrial right-to-left shunt, a portion of the injected microbubbles are likewise not removed by the lungs.

While it is known that a portion of the injected microbubbles can be detected in cranial arteries using transcranial Doppler systems (which are expensive and require advanced training), Applicants have found that a portion of the injected microbubbles also can be detected in peripheral arteries (e.g., brachial arteries) using a Doppler detector. The presence of the micro-bubbles in arterial blood is anomalous since, under normal conditions, the lungs remove these micro-bubbles, i.e. air emboli, as de-oxygenated blood undergoes re-oxygenation. These micro-bubbles are air suspended in a saline solution as known in the art of agitated saline contrast echocardiography.

It is known that some patients with little or no shunt during normal breathing can have significant shunts provoked by special breathing techniques or other stressors. For example, a Valsalva maneuver, specifically the release phase, is know to cause some right-to-left atrial shunts to open. The initiation and termination of breathing techniques such as a Valsalva maneuver may be guided by software in the control system (16). The system and method utilizes the release phase of the Valsalva maneuver to induce shunting. The patient may be instructed to perform a Valsalva maneuver beginning prior to or at about the time of microbubble injection. The Valsalva maneuver is then stopped when the microbubbles are expected to reach the right atrium, as it is an atrial shunt in some patients will open when the Valsalva maneuver is ceased. The system may provide a visual and/or audible indication of when the patient should commence and release the Valsalva maneuver based on, for example, a predetermined period of time after microbubble injection.

A pressure sensor may also be used to ensure that the patient performs a Valsalva maneuver with adequate pressure and/or to provide timing data to the control system (16) indicating when the Valsalva maneuver is commenced and released. By way of example, the patient may be instructed to blow into a disposable mouthpiece that includes a pressure transducer in communication with control system (16). Adequate pressure may be indicated audibly and/or visually, such as by a graphic display visible to the clinician and/or patient. In the embodiment shown in FIG. 2, a mouthpiece (60) having a pressure transducer (62) is depicted in communication with signal processing unit (20) of control system (16). Optimal timing of saline injection, Valsalva release (at a time when bubbles are expected to reach the right atrium), and detection (when bubbles are expected to arrive in the radial artery) is coordinated by the software running in the control system (16) (e.g., in signal processing unit (20)). Saline injection and Valsalva initiation and release may be prompted by sensory cues, e.g. visual and/or auditory.

One or more software programs resident in memory and controlled by one or more computer processors in control system (16) may be used to synchronize each step of the procedure. As mentioned previously, control system (16) may comprise at least one memory storing one or more software programs, and at least one processor for running the software programs. At least one display may be operatively connected to control system (16) for displaying information to a clinician such as instructions for performing a cardiopulmonary defect detection method and/or displaying the results of such defect detection method. As used herein, the term processor is intended to encompass any of a variety of processors or processor-like electronic components that can read and run a set of machine-readable instructions (i.e., software, programs, algorithms, etc.) such as, for example, computers, PICs, microprocessors, microcontrollers, and the like.

Timing algorithms in the software allow the system to begin to acquire baseline signal data from the target artery at the appropriate time before the arrival of microbubbles, which provides an internal control. For example, a clinician may position the transducer (12) adjacent the target artery and then provide input (e.g., using keyboard (26)) to control system (16) that the patient is ready for testing. Control system (16) will then collect background Doppler echo data for a predetermined period of time (“baseline phase”) while the patient remains stationary and quiet. Thereafter, control system (16) provides a visual and/or audible cue to the clinician to inject the microbubble dispersion. The quantity of agitated saline may be controlled, as well as the severity of agitation. A signal that the injection has taken place may then be provided to control system (16), such as by the clinician pressing switch (52) on the syringe used to inject the microbubbles. In an alternative embodiment, the injection can be accomplished automatically under the control of control system (16). Control system (16) may also signal (visually and/or audibly) that the patient should begin a Valsalva maneuver. Similarly, control system (16) may also signal (visually and/or audibly) that the patient should release the Valsalva maneuver after a predetermined period of time.

The control system (16) will continue to acquire Doppler echo data after the baseline phase (the “detection phase”) for a predetermined period of time after injection (i.e., until after a point in time when bubbles are expected to reach the artery being monitored). The control system (16) compares signals collected during the “baseline” and “detection” phases, and then calculates the mathematical probability that a shunt such as a PFO, ASD or PAVM is present or absent. The Doppler signal data is mathematically modified to minimize or remove the background noise and to enhance detection of peaks that depart significantly from the background.

In one embodiment, Doppler unit (18) processes Doppler echo data received by transducer (12) and generates an audio signal which signal processing unit (20) then processes and analyzes using one more various algorithms. The signal processing unit (20) of control system (16) identifies transient signals that indicate increased reflectance due to single or clustered bubbles passing through the ultrasound beam emitted by transducer (12). Signal processing may incorporate noise, specularity and/or envelope filters that enhance the difference between background noise and bubble transients. Wavelet transforms and Fourier transforms are known to be useful means to resolve signals of interest from the background, and may be used for this purpose as well.

One such statistical model requires the determination of the Gaussian distribution using a calibration range (e.g., the 4 minutes preceding the first detection of possible signal artifacts from the detection of microbubbles) to determine the mean and variance before microbubble detection and subtract the background contribution from each potential detection peak, and simple background subtraction utilizing microbubble free signals. Various commercially available software packages which create statistical models, e.g. MATLAB, can be utilized to create statistical models which transform signal data into useful information. The qualitative magnitude of the shunt (roughly proportional to the total number of bubbles detected) may also be calculated. In an alternative embodiment, one or more mathematical modeling algorithms may be incorporated into an application or program resident on a handheld device such as an iPhone or Windows CE based device to evaluate the signal and eliminate or minimize the background (including artifacts) while maximizing microbubble sensitivity. In such an arrangement, the device provides signal processing unit (20).

Signal intensity data (e.g., an audio signal from Doppler unit (18)) may be recorded over time and analyzed by algorithms written in machine readable code (i.e., software) stored in computer readable media as part of control system (16). Bubble detections can be depicted as brief spikes in intensity that exceed a threshold signal intensity established during the baseline calibration phase. The signal is processed to minimize false positives from signal artifacts from things such as atheromas, platelets, and thrombi. Microbubble detections can also be denoted in real time by an audible chirp and/or by graphical representation of Doppler detector response. Plots of signal intensity versus time can be depicted on a display (such as an LCD screen) by the control system (16). Such a display provides recognizable, real time feedback to the operator. Interpretation of this data is provided through interpretation algorithms that analyze the collected data and dependent variables. The software calculates a numerical probability statement to indicate whether a shunt is likely present or absent. The control system may also report the magnitude of any shunt that is present with a probability >0.95. All of this information may be displayed to the user on a display operatively connected to control system (16) and/or provided in a report generated by control system (16) and subsequently printed.

Raw and processed data useful for the characterization of cardiopulmonary defects include time of onset of the first detection of a bubble, the duration of bubble detections, the cumulative area under detected transient Doppler ultrasound peaks attributed to injected bubbles, the number detected transient Doppler ultrasound peaks attributed to injected bubbles, the spacing of detected transient Doppler ultrasound peaks attributed to injected bubbles, metrics related to the Doppler pulse signature, and metrics related to the curve fitted across detected transient Doppler ultrasound peaks attributed to injected bubbles. This listing of variables is non-exclusive and intended as an example of the kinds of data that can be extracted from the raw and processed signal that are useful in characterizing such defects based on empirical data collected from patients with known or suspected defects stored for use by the control system. In other words, the control system (16) compares these acquired variables to empirical data stored in memory in order to determine, for example, the presence, type and/or magnitude of a defect.

As previously mentioned, the control system (16) analyzes the signal data to minimize the effect of the background (e.g., blood cells traveling through the artery being monitored) and to increase the instrument sensitivity to the reflection from the micro-bubbles. The control system (16) then generates a result from its analysis and reports its assessment of the probability that a cardiopulmonary defect, e.g. a right-to-left shunt, is present or absent.

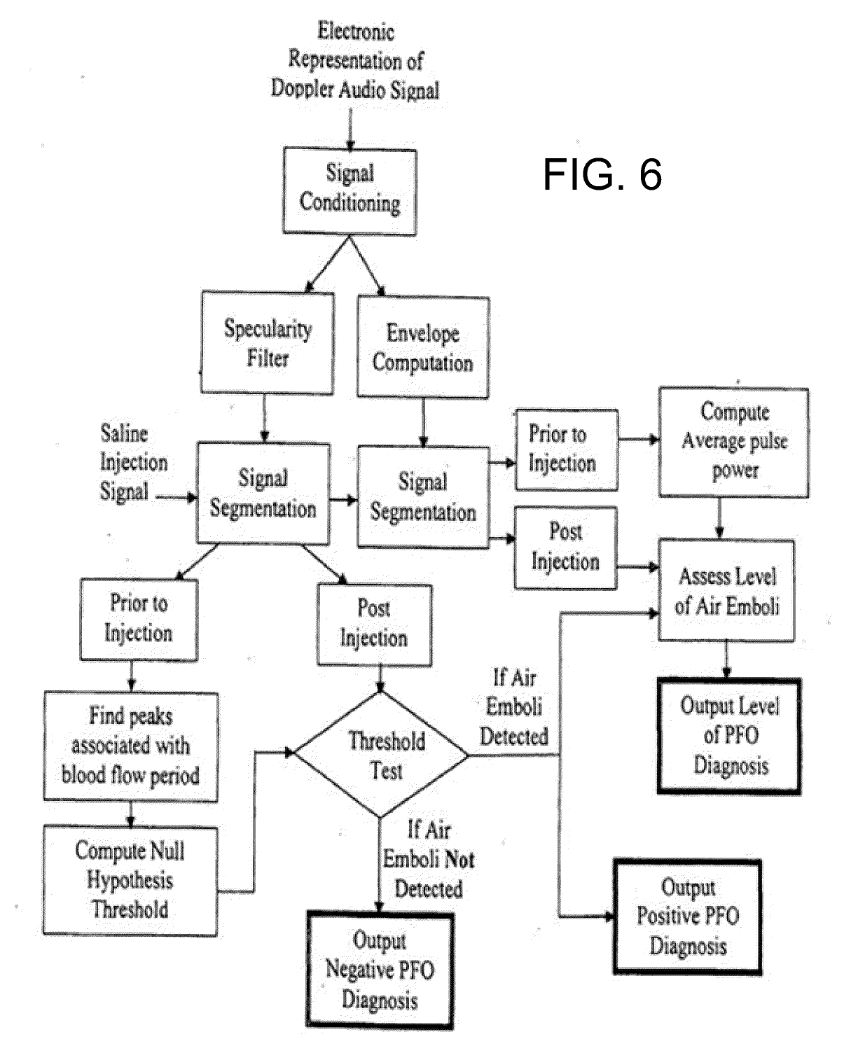

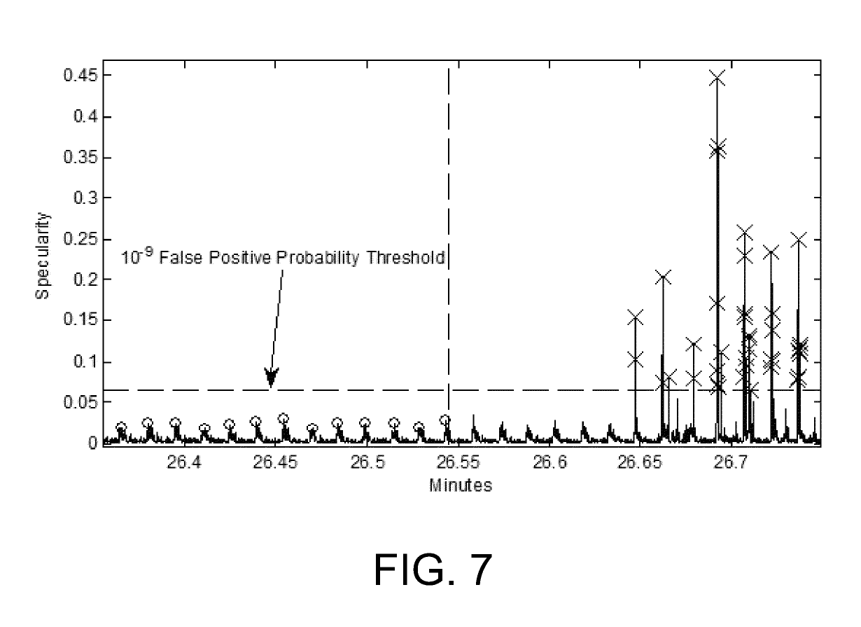

Prior to the injection, data is collected (e.g., for a minute or more) and used to compute the statistics of the peaks from the normal Doppler signal after specular filtering and/or envelope detection. A maximum value is found in the first few seconds of the baseline to mark the location of the first pulse cycle (i.e., Doppler reflections from blood cells traveling through the artery being examined). The distance to the next pulse is estimated with a local autocorrelation. A search for a maximum around this shifted region is performed by computing a local maximum and this point is used for identifying the next cycle. This is repeated until the microbubbles are injected into a vein (a “cue point”). The peaks in the baseline are used to compute the statistics for the null case (no microbubbles detected). A Gaussian, Rayleigh, Rician or similar continuous distribution assumption of probability density is used to model the variations in the peaks based on the computed mean and variance. The distribution is then used to compute the threshold for a given false positive probability. The algorithm outlined in FIG. 6 illustrates this process wherein a specularity filter is applied in order to determine a false positive probability threshold. A sample plot of the output of the specularity filter is shown in FIG. 7, wherein the “∘” markers denote the detected pulse peaks in the baseline data. The mean and variance are computed from these when a Gaussian distribution is used to compute the detection threshold, denoted by the horizontal dashed line in FIG. 7. The broken vertical line in FIG. 7 denotes the time of microbubble injection (the cue point). Specularity filter outputs exceeding the detection threshold in the detection phase (to the right of the cue point) are labeled as detections at the “X” markers.

The algorithm depicted in FIG. 6 processes the audio output of the Doppler unit (18). In one embodiment, Doppler unit (18) may comprise a conventional Doppler flow meter capable of generating an audio output representing Doppler echo signals received by the transducer (by demodulating continuous Doppler data down to the audio range). Signal conditioning is performed on the audio output in order to reduce noise and artifacts. Conditioning can be built into the transducer, Doppler unit (18) and/or A/D hardware. By way of example, an anti-aliasing filter may be used for signal conditioning. Further filtering can be performed either with dedicated hardware or with control system software to suppress output of band noise and artifacts. In one embodiment, data processed using the audio output of a conventional Doppler flow meter may be filtered to remove signal energy below about 100 Hz (which may be dominated by artifact such as sensor motion on the patient's skin), and above about 2000 Hz (which may be dominated by white noise).

One aspect of the algorithm depicted in FIG. 6 is the application of a specularity filter as part of the transient feature extraction to enhance the detection of transient components in a pulsating flow background typical of air emboli echoes. One challenge to reliably detecting transients is limiting the false positive caused by the pulsating background signal. While this signal is quasi-periodic and level changes can be predicted to some degree, the rate can change due to patient response. For most tracking and adaptive algorithms this results in transitory artifacts until the algorithm converges on the new patterns. To avoid this problem, the specularity filter operates on short time intervals or window lengths (e.g., about 1 to about 50 ms) over which the echo from a dominate air emboli scatterer appears in the Doppler signal. While the leading edges of pulsating blood cells also create a transient echo, the many scatterers involved create diffuse echoes that have lower specularity. In other words, the transient is not as sharp as when air emboli are present in the ultrasonic field. Thus, by applying a specularity filter to the audio output of the Doppler unit (18) in the signal processing unit (20), microbubbles in the target artery may be readily identified as peaks exceeding the false positive probability threshold (shown as “x” markers in FIG. 7).

The envelope of the continuous wave (“CW”) Doppler signal also shows higher levels of backscatter power when air emboli are present and can be used in conjunction with multiple techniques for interpreting the transient signals including wavelet analysis, higher order statistical analysis, and gradient/peak properties for the purpose of assessing a level of confidence in the decision or the severity of the shunt. In the embodiment depicted in FIG. 6, if air emboli (i.e., microbubbles) are detected after application of a specularity filter, envelope detection on the same audio output may be used to assess the confidence level that a defect (e.g., a right-to-left shunt) is present, the type of defect (e.g., PFO, ASD or PAVM), the size or extent of the shunt, and/or other parameters related to the defect. In some instances, envelope detection alone may be used to diagnose the presence of a shunt, particularly when a large density of air emboli is detected. It should also be noted that, although FIG. 6 references the detection and characterization of a PFO, the same algorithm may be used to detect and characterize other types of right-to-left shunts or even to distinguish between different types of shunts (e.g., to distinguish a PFO from an ASD).

The algorithms for specularity filtering and envelope detection use knowledge of the injection time to segment the input signal into baseline and test (also referred to herein as detection) zones. The false positive probability threshold is computed from the transient features extraction from the baseline data using a null hypothesis test. A predetermined or user-specified false positive rate is used by the algorithms in conjunction with statistics computed from the baseline data to compute the threshold. If the same transient features extracted from the test segment zone exceed the threshold, air emboli detection is positive. If positive, further analysis and interpretation of the signal will be performed to assess the confidence level of positive detection and severity of the shunt. By way of example, a simple counting of detected peaks in the data set following specularity filtering and/or envelope detection may be used.

Specularity Filter:

A specularity filter may be based on the generalized spectrum and has been applied in other ultrasonic tissue characterization and non-destructive testing applications. The specularity filter exploits phase information in the Doppler signal as well as the power over short-time window analysis. The specularity filter is sensitive to phase patterns in the Discrete Fourier Transform (DFT) consistent with sharp isolated transients/echoes. This property fits well with detecting microbubbles in a peripheral artery using Doppler ultrasound, as the specularity filter suppresses the dense scattering of ultrasound from blood cells and as well as from weak scattering emboli, while enhancing the sharp and isolated transients typical of air emboli (microbubbles) in blood. The specularity filter windows the signal into overlapping segments, with a typical segment length between about 1 to about 100 milliseconds. The specularity filter computes the DFT and performs a complex autocorrelation of a single sided spectrum (positive frequencies). Note that this is an autocorrelation in the frequency domain, not the usual time domain. The sum of the absolute value of correlated DFT coefficients are taken as the output of the filter. The short-time window is slid along over the entire signal and computes the detection statistic associated with the signal in that window.

More specifically, for the specularity filter the fast fourier transform (“FFT”) of each windowed signal is taken with zero padding equal to the lowest power of 2 greater than 2N., wherein N is the window length (e.g., between about 1 and about 50 ms. The FFT coefficients corresponding to the positive frequencies are extracted, and this segment of complex numbers is denoted as \hat{X}ₛ. Then the collapsed average is computed by the autocorrelation of this segment given by:

$$C ( λ ) = {\sum }_{k = {F}_{l}}^{{F}_{h}} {\hat{X}}_{s} ( k - λ ) {\hat{X}}_{s}^{*} ( k )$$

where Fₗ and Fₕ are the FFT indices for frequencies corresponding to 100 and 2500 Hz, and superscript * denotes the complex conjugate. The index λ represents the lag between frequencies for the autocorrelation. Finally to obtain the statistics used in the null hypothesis test, the area under the collapsed average magnitude is taken to obtain the specularity parameter:

$$S = {\sum }_{λ = L}^{H} C ( λ )$$

Where L and H are the indices over which the area under the collapsed average curve is taken. The lower index L may be computed from the window length and zero padding to remove the impact of windowing artifacts (e.g., twice the FFT length divided by N). These indices determine the frequency lag range which can be tuned to the signal properties. If desired, the entire lag range may be used.

For envelope detection, any of a variety of techniques may be used. By way of example, the Hilbert transform may be performed on each window to form the analytic signal:

yₐ=x+j\hat{x}

where x is the windowed signal of length N, the hat denotes its Hilbert transform, and j is the complex number square root of −1. The envelope is then the magnitude of yₐ:

Y=|yₐ|=\sqrt{x²+\hat{x}²}

The computed envelope signal may then be resampled, such as down to 100 Hz. The null hypothesis may be applied to the envelope signal for purposes of establishing the false probability threshold (this may also be performed on the output of the specularity filter for the same purpose).

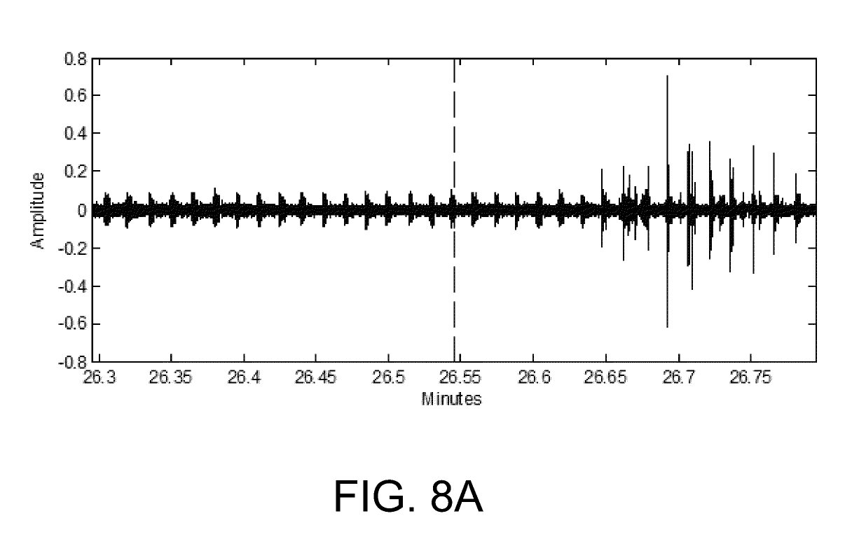

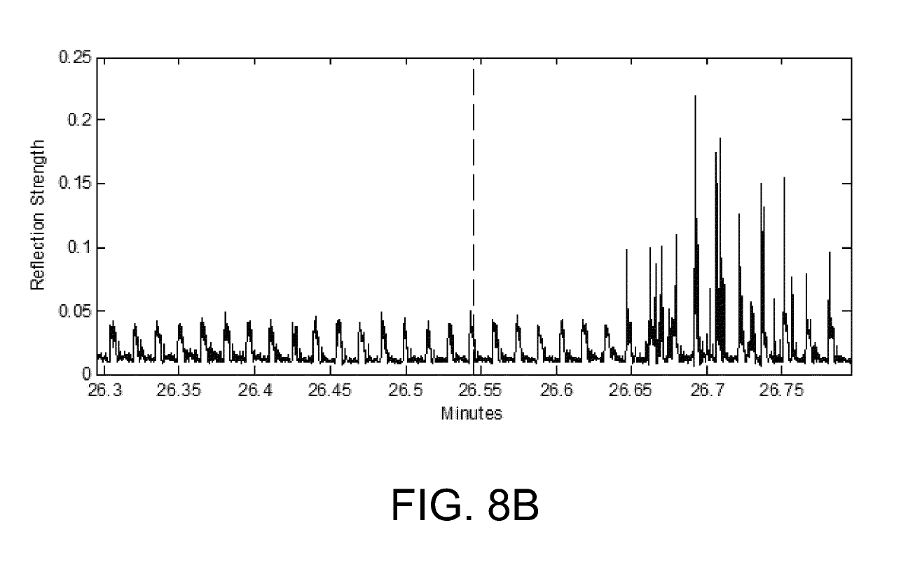

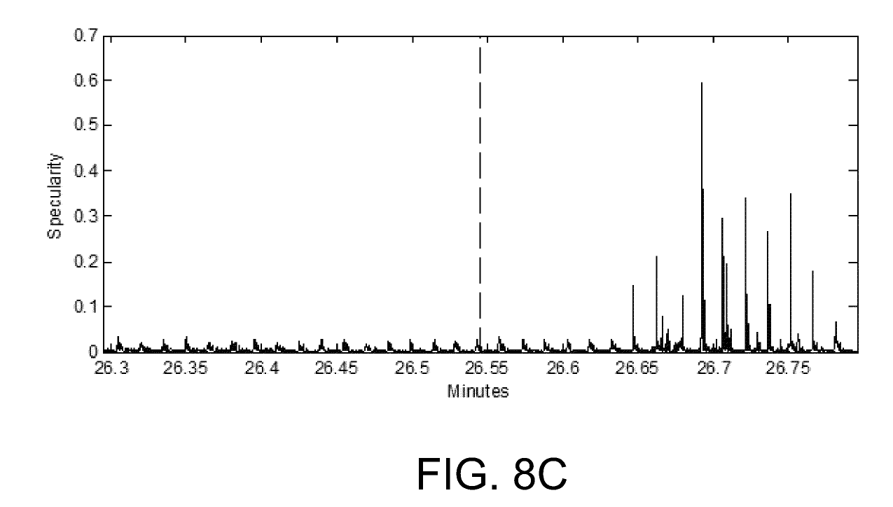

As further discussed herein, FIG. 8A depicts an exemplary audio output from a Doppler flow meter. FIG. 8B depicts the envelope signal for the audio output of FIG. 8A, and FIG. 8C depicts the specularity filter output. In all three plots of FIG. 8, the vertical dashed line indicates the time of microbubble injection. As will be noted, the specularity filter increased the magnitude of the bubble echoes relative to the regular blood flow echoes. This increase improves sensitivity for a fixed false positive rate.

In the examples described below, the Doppler unit (18) was an Ultrasonic Doppler Flow Detector Model 811-B, manufactured by Parks Medical Electronics, Inc., Aloha, Oreg. Its audio output was sampled using an Edirol R-4 Four-Channel Portable Recorder (Roland Corporation) and sampled at 44.1 kHz. Comparisons of specularity filtering with other blood flow signal representations. Broken vertical line indicates the time of agitated saline injection. The specularity filter and envelope detection algorithms described above were implemented in MATLAB (Mathworks, Natick, Mass.) on a laptop computer. It is contemplated that the algorithms be ported to Lab VIEW for a real-time PC implementation using, for example, a USB data acquisition device. For compact devices the algorithm can be ported to a digital signal processing (DSP) chip, such as the Texas Instruments Series TI TMS320C6000 DSP (C6711, C6713, C6416) or a Field Programmable Gate Array (FPGA), such as Xilinx (San Jose, Calif.) Virtex-II and V FPGA. Both devices can support efficient code export by MATLAB with their Real-Time Embedded Toolboxes.

### Example 1

A Doppler transducer was positioned over a patient's radial artery and Doppler ultrasound data was collected for a period of time to establish baseline data. Thereafter, a microbubble dispersion was injected into a vein of the patient while Doppler data was continued to be collected. FIG. 8A depicts the audio output from the Doppler flow meter, wherein the vertical dashed line indicates the time of injection. FIG. 8B depicts the envelope signal for the audio output of FIG. 8A, and FIG. 8C depicts the specularity filter output using a 10 ms window. As noted from FIG. 8C, the specularity filter increased the magnitude of the bubble echoes relative to the regular blood flow (baseline phase) echoes. This increase improves system sensitivity for a fixed false positive rate. FIGS. 8B and 8C also demonstrate the existence of a significant right-to-left shunt in the patient.

FIG. 7 depicts the computation of the false positive probability threshold for this same data set using the output of the specularity filter. A Gaussian assumption was used to model the variations on the local peaks in the baseline phase (pre-injection) based on the computed mean and variance of the locally maximum feature peaks so that the highest peaks in every pulse are used. The error function was then used to compute the threshold for a false positive probability of 10⁻⁹. The “∘” markers in FIG. 7 denote the detected pulse peaks in the baseline data, and the horizontal line identifies the computed detection threshold. Specularity filter outputs exceeding this threshold during the detection phase (post-injection) are labeled as microbubble detections with “X” markers in FIG. 7. Similar calculations may be made using the envelope signal output.

### Example 2

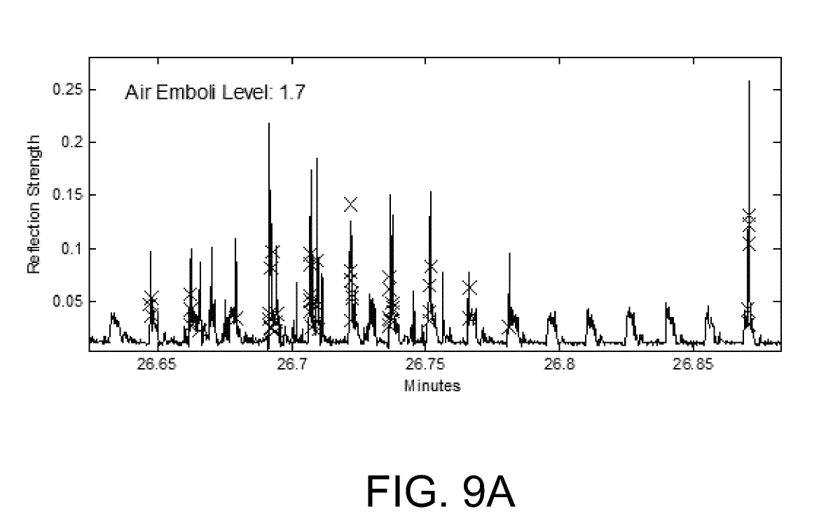

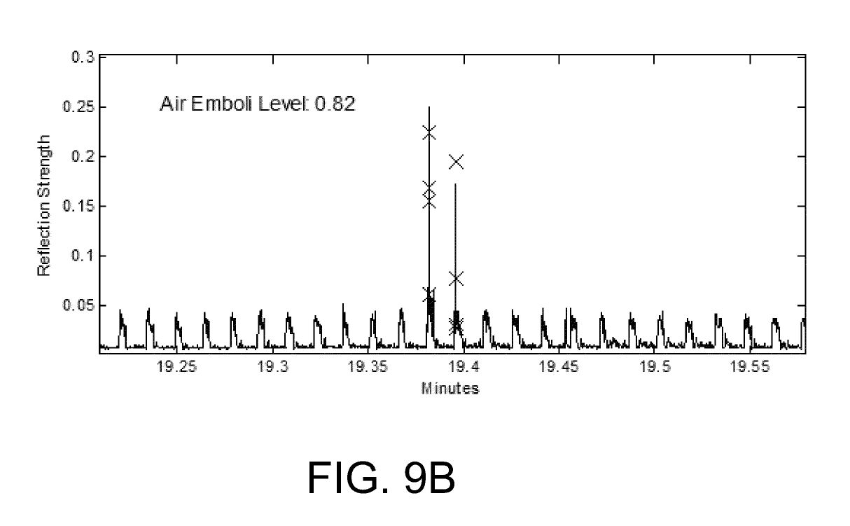

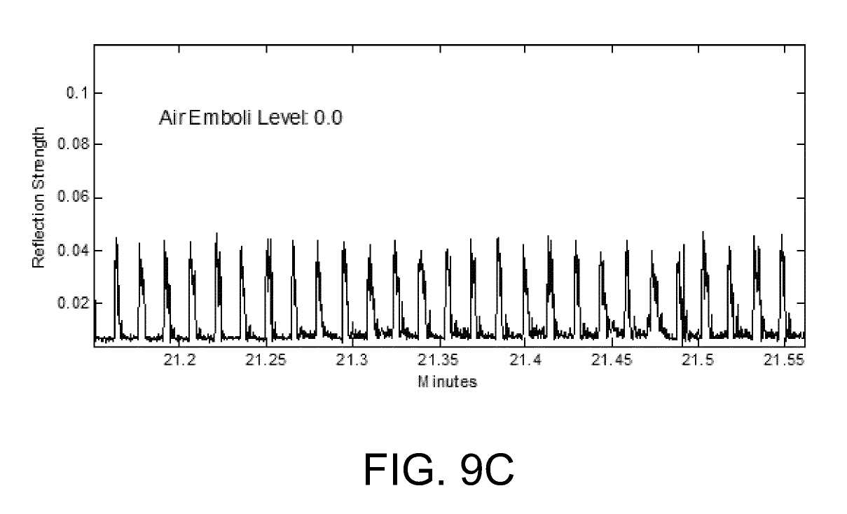

In this example, FIGS. 9A-C depict air emboli level assessments on signal envelope with false positive detection probability set to 10⁻⁶. “X” markers denote corresponding detections from the specularity filtered signal. FIG. 9A shows a high-level of air emboli, FIG. 9B a mid-level of air emboli, and FIG. 9C no emboli detected.

Having shown and described various versions in the present disclosure, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, versions, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

What is claimed is:

1. A diagnostic system for detecting a right-to-left cardiopulmonary shunt in a patient, comprising:
at least one Doppler ultrasound transducer;
a transducer housing;
a transducer housing placement device to affix said Doppler ultrasound transducer housing over a peripheral blood vessel so that said at least one Doppler ultrasound transducer can remain stationary over said blood vessel during transmission of ultrasound and receipt of ultrasonic echoes; and
a control system configured to receive and process said echoes and transform said echoes into data indicative of the presence or absence of a bubbles within the peripheral blood vessel, thereby determining whether or not the patient has a right-to-left cardiopulmonary shunt.

2. The diagnostic system of claim 1, wherein said placement device is configured to affix said housing over an artery in a patient's wrist.

3. A method of detecting a right-to-left cardiopulmonary shunt in a patient, comprising the steps of:
(a) positioning a Doppler ultrasound transducer over a peripheral artery in the patient;
(b) collecting Doppler ultrasound data comprising Doppler echoes from the patient's peripheral artery;
(c) injecting microbubbles into a vein in the patient while continuing to collect said Doppler ultrasound data; and
(d) determining the presence of right-to-left cardiopulmonary shunt in the patient by comparing collected data from before and after the injecting step.
